(19) United States
(12) Patent Application Publication
(10) Pub. No.: US 2013/0204217 A1
(43) Pub. Date: Aug. 8, 2013

(54) HYDROGEL MATRIX HAVING INCREASED ABSORPTION CAPACITY FOR LIQUIDS

(71) Applicants: Martin Junginger, Hermaringen (DE); Julie Horny, Bollwiller (FR)

(72) Inventors: Martin Junginger, Hermaringen (DE); Julie Horny, Bollwiller (FR)

(21) Appl. No.: 13/516,570

(22) PCT Filed: Dec. 14, 2010
(86) PCT No.: PCT/EP10/07600

§ 371 Date: Aug. 9, 2012

(30) Foreign Application Priority Data
Dec. 24, 2009 (EP) 09016006.0

(51) Int. Cl. A61F 13/534

(52) U.S. Cl. CPC A61F 13/534
USPC 604/368

(57) ABSTRACT

The invention relates to a multilayer wound dressing, in particular for moist wound treatment, comprising a first layer as an absorbent layer, which comprises a hydrogel matrix, and at least one second layer, which is applied to the first layer on the side facing away from the wound. The hydrogel matrix comprises 54 to 60 wt % of propylene glycol, a pre-polymer having isophorone diisocyanate end groups and a diamine based on polyethylene oxide in a quantity of a total of 38 to 42 wt %, 0 to 5 wt % of an inorganic chloride, and the remainder water, wherein the ratio of the reactive groups of isocyanate to the amine groups of the diamine is intended to be 1.25 to 1.35.

This invention concerns wound dressings particularly as wound treatment means in the granulation and epithelization phase. These wound dressings are useful for moist treatment of wounds in particular.

The healing of skin wounds is based on the ability of the skin to regenerate epithelium and also connective and supporting tissue. Regeneration itself is characterized by a complex interplay of overlapping cellular activities which advance the healing process step by step. Three essential healing phases of a wound have been described in the literature irrespective of the type of wound. They include the inflammatory or exudative phase for blood coagulation and wound cleaning (phase 1, cleaning phase), the proliferative phase for building granulation tissue (phase 2, granulation phase) and the differentiation phase for epithelization and scar formation (phase 3, epithelization phase).

Numerous proposals for augmenting the individual wound healing phases are described in the literature. Especially wound dressings comprising hydrogels have for some time been the subject matter of numerous articles in the technical literature and also of patent documents. The European patents EP 455 324 B1, EP 528 091 B1, EP 567 704 B1 or EP 630 629 B1, for instance, describe transparent hydrogel wound dressings having various constructions. These sometimes multilayered wound dressings comprise a water-containing or dehydrated hydrogel as wound contact layer for the treatment of burn wounds.

EP0426422B1 discloses a wound dressing comprising a hydrogel based on a polyurea-polyurethane copolymer. The compositions described contain 15 to 30 wt % of a polyhydric alcohol. The ratio for reactive groups of isocyanate to amine groups in the hydrogel compositions described in EP0426422B1 is in the range from about 0.63 to 1.40. The ratio is 1.23 in an embodiment said to be preferred. A wound dressing which contains a hydrogel according to the compositions described in EP0426422B1 is on the market under the designation Hydrosorb® (Paul Hartmann AG, Germany). The wound dressing is capable of absorbing about twice its own weight of liquid within a period of 48 hours.

Furthermore, the international applications WO 02/38 097 A1, WO 02/47 761 A1, WO 03/011 352 A1, WO 03/086 255 A1, WO 2004/052 415 A1 or EP 1 658 865 A1 describe wound dressings comprising a hydrogel and a polymer foam.

The applicant's application for a patent under application number DE102008031183.9, which constitutes state of the art within the meaning of EPC Article 54(3), likewise describes a multilayered wound dressing having a wound contact layer as first layer and at least one second layer as absorbent layer which comprises a hydrophilic foam of polyurethane. The wound contact layer can be a hydrogel based on a polyurea-polyurethane copolymer. The operative example of DE102008031183.9 discloses a hydrogel comprising 17.5 wt % of propylene glycol, while the ratio for reactive groups of isocyanate to amine groups in the hydrogel described is 1.23.

State-of-the-art wound treatment requires wound dressings which speed the healing of wounds and augment the natural wound healing process. Suitable wound dressings have to combine different desirable properties. In addition to having very good skin and tissue compatibility, a wound dressing shall ensure the moist milieu which promotes wound healing, while excess liquid should be absorbed. A wound dressing shall further have atraumatic properties, i.e., the wound dressing must be removable when it needs to be changed, without damaging newly grown wound tissue. A moist milieu is beneficial for wound healing, although an alkaline pH, especially above pH 8, should be avoided. At the same time, excess liquid shall be transported out of the wound region and be absorbed by the wound dressing. Excess liquid or exudate can otherwise lead to maceration of the edge of the wound. In practice, therefore, the users, such as physicians or care personnel, are frequently desirous that a wound dressing has a very high absorption capacity.

In view of the described and commercially available wound dressings having a polyurea-polyurethane copolymer hydrogel matrix, the present invention has for its object to provide an improved wound dressing. The present invention more particularly has for its object to provide an improved wound dressing useful for wound healing in the granulation and/or epithelization phase. It is a further object of the present invention to provide an atraumatic wound dressing which influences the pathological state of a wound such that a normal, natural wound healing process can occur. To this end, the wound dressing shall provide a sufficient amount of moisture to the wound and at the same time have a wound-compatible pH close to neutral. It is more particularly desirable for the wound dressing to have good absorptive capacity for liquids.

These objects are achieved by a multilayered wound dressing as proposed in claim 1. A multilayered wound dressing according to the present invention accordingly comprises a first layer as absorbent layer comprising a hydrogel matrix, and at least one second layer, applied atop the first layer on the wound-remote side, characterized in that the hydrogel matrix comprises from 54 to 60 wt % of propylene glycol, altogether from to 42 wt % of a prepolymer having isophorone diisocyanate end groups (hereinafter referred to as “isocyanate”) and of a diamine based on polyethylene oxide and from 0 to 5 wt % of an inorganic chloride, balance water, wherein the ratio for reactive groups of isocyanate to amine groups of diamine shall be in the range from 1.25 to 1.35.

The second layer can be a further absorbent layer, a liquid distribution layer or a backing layer.

The proposed multilayered wound dressing rests on the surprising discovery that a hydrogel matrix having, compared with prior art polyurea-polyurethane copolymer hydrogel matrices, a significantly increased absorption capacity for liquids and also a pH promotive of wound healing is obtainable when the hydrogel matrix comprises a composition which

a) comprises propylene glycol as polyhydric alcohol,

b) comprises a specifically selected amount of propylene glycol,

c) a prepolymer having isophorone diisocyanate end groups and a diamine based on polyethylene oxide in a specifically selected proportion, while

d) a certain ratio (“NCO:NH2”) for reactive groups of isocyanate (“NCO”) to amine groups of diamine (“NH2”) has to be present.

The composition further comprises from 0 to 5 wt % of an organic chloride, balance water.

A wound dressing according to the present invention and comprising a hydrogel matrix of this type may also display tack to the wound site and the surrounding skin, and this may facilitate applying the bandage.

A hydrogel matrix comprising from 54 to 60 wt % of propylene glycol, altogether from 38 to 42 wt % of a prepolymer having isophorone diisocyanate end groups and a diamine based on polyethylene oxide, from 0 to 5 wt %, preferably from 0.5 to 1.5 wt %, of an inorganic chloride, balance water, wherein the ratio for reactive groups of isocyanate to amine groups of diamine is in the range from 1.25 to 1.35, has a near-neutral pH augmentative of natural wound healing, for example a pH of 7.48. The hydrogel matrix further has a high absorption capacity for liquids. For instance, 5.2 times it own weight of demineralized water can be absorbed within 48 hours. It was likewise found that the hydrogel matrix, when it is in direct contact with the wound, possesses a degree of tack to the wound site. Tack with regard to the wound site is desirable because this facilitates applying the bandage. Tack with regard to the wound and the surrounding skin was assessed on a relative scale from 1 to 5 in the course of the extensive experimentation conducted in connection with the present invention. It was assessed on the basis of the subjective impression of one person by comparison with the tack of various products as perceived between the index finger and the gel. Commercially available products were put into 5 categories by one person. A tack of 1 on the scale indicates that the wound dressing does not adhere to healthy skin, while a tack of 5 indicates a very high level of tack. A tack of 5 is less desired because a wound dressing which adheres that strongly can lead to the edges of the wound becoming damaged as the bandage is changed for example. The hydrogel matrix disclosed in the present invention can have a low level of tack, for example a tack with a value of 2.

To obtain the desired properties on the part of the hydrogel matrix, it is essential that the composition of the hydrogel matrix comprise firstly a propylene glycol content of 54 to 60 wt %. This is a significantly higher propylene glycol content compared with the compositions customarily described in the prior art. Secondly, the composition shall comprise altogether from 38 to 42 wt % of a prepolymer having isophorone diisocyanate end groups and of a diamine based on polyethylene oxide, subject to the proviso that the ratio for reactive groups of isocyanate to amine groups of diamine adjusted to the range from 1.25 to 1.35 must be present. It was found that any departure from the composition disclosed in the invention provides a hydrogel having comparatively inferior properties, for example a hydrogel having an undesirably high pH of more than 8, a hydrogel having insufficient absorption capacity or a hydrogel that does not cure sufficiently. Particularly advantageous hydrogels are obtainable when the composition comprises from 54 to 60 wt % and preferably from 56 to 58 wt % of propylene glycol and altogether from 38 to 42 wt % and preferably from 39 to 41 wt % of a prepolymer having isophorone diisocyanate end groups and of a diamine based on polyethylene oxide, provided the ratio for reactive groups of isocyanate to amine groups of diamine is adjusted to be in the range from 1.27 to 1.33 and ideally in the range from 1.29 to 1.31. The composition further comprises from 0 to 5 wt %, preferably from 0.5 to 1.5 wt % and ideally 0.9 wt %, of an inorganic chloride, which is preferably sodium chloride. Hydrogels of this type have outstanding properties with regard to absorption capacity, pH and stability. The hydrogels can also have tack to the skin, as is desired when the hydrogel is used as wound contact layer.

The hydrogel is obtainable by mixing water, propylene glycol and NaCl and then adding the molten amine under agitation until a homogeneous solution has formed. The isocyanate is then added. The gel is very quick to cure, typically within a few minutes, for example within 10 minutes. Curing, if desired, can be retarded by cooling the composition after the isocyanate has been added. The ratio for reactive groups of isocyanate to amine groups of diamine can be computed and adjusted in the usual manner from the molecular weights of the starting materials used having regard to their purity. For very accurate adjustment of the ratio for reactive groups of isocyanate to amine groups of diamine it is advantageous for the amine content of the mixture of water, propylene glycol, NaCl and diamine to be accurately measured before the isocyanate is added. Conventional analytical methods can be used for determining the amine content. The ratio for reactive groups of isocyanate to amine groups of diamine can be adjusted to a value in the range from 1.25 to 1.35, preferably in the range from 1.27 to 1.33 and ideally in the range from 1.29 to 1.31 via the amount of isocyanate added and having regard to the amine content measured. Particularly advantageous gels are obtained when the ratio for reactive groups of isocyanate to amine groups of diamine is adjusted to 1.30.

A particularly suitable first layer for the multilayered wound dressing of the present invention has a hydrogel matrix having the following composition:

57 wt % of propylene glycol,

15.3 wt % of a diamine based on polyethylene oxide, for example Jeffamine ED-2003,

0.9 wt % of NaCl,

24.7 wt % of an isocyanate, for example Aquapol PI-13000-3,

balance water.

The ratio for reactive groups of isocyanate to amine groups of diamine in the composition is 1.30.

A multilayered wound dressing of the present invention, comprising the hydrogel matrix and at least one second layer, applied atop the first layer on the wound-remote side, has an unexpectedly high absorption capacity for liquids.

An embodiment of the multilayered wound dressing comprising a hydrogel matrix of the aforementioned composition in a thickness of 0.8 mm and a second layer configured as backing material is capable of absorbing about 5.2 times its own weight of water within 48 hours. The hydrogel composition here comprises 57 wt % of propylene glycol, about 24.7 wt % of Aquapol FI-13000-3 isocyanate, 15.3 wt % of Jeffamine ED-2003 diamine based on polyethylene oxide, 0.9 wt % of NaCl and balance (about 3 wt %) of water. The ratio for reactive groups of isocyanate to amine groups of diamine is set to 1.30. The ratio for reactive groups of isocyanate to amine groups of diamine can be computed and adjusted in the usual manner from the molecular weights of the starting materials used having regard to their purity. For very accurate adjustment of the ratio to the value of 1.30 it is advantageous for the amine content of the solution of water, propylene glycol, NaCl and diamine to be determined and for the amount of isocyanate to be appropriately conformed thereto. A minor difference may become apparent compared with the aforementioned amount of isocyanate, for example 24.7 wt % of Aquapol. A wound dressing comprising such a hydrogel matrix has a pH of 7.48, which is supportive of wound healing. The wound dressing further has low skin tack of 2.

The at least one second layer of the multilayered wound dressing is a further absorbent layer, a liquid distribution layer or a backing layer. Preferably, the at least one second layer is a backing layer. This backing layer can consist of various materials. Typically, wound dressings utilize textile backing materials, nonwovens, polymer films or polymer foams. This backing layer may be in direct or indirect contact with the first layer, which comprises a hydrogel matrix. In the case of direct contact, the backing layer is laminated directly onto the hydrogel matrix, whereas in the case of indirect contact, the backing layer is applied atop the hydrogel matrix by means of an adhesive. This adhesive may be applied between the backing layer and the absorbent layer in a uniform manner or merely in sub-regions.

The backing layer of a wound dressing that is in accordance with the present invention may utilize in particular polymer films or polymer foams. Very particular preference is given to polymer films or polymer foams which are water impermeable and have a high moisture vapor permeability. Films or foams particularly suitable for this are fabricated from polyurethane, polyester, polyetherurethane, polyesterurethane, polyether-polyamide copolymers, polyacrylate or polymethacrylate. More particularly, a water impermeable and moisture vapor permeable polyurethane film or a water impermeable and moisture vapor permeable polyurethane foam is suitable for use as backing layer. More particularly, a polyurethane film, polyesterurethane film or polyetherurethane film is preferable for use as polymer film. However, very particular preference is also given to polymer films from 15 to 60 μm, more particularly from 20 to 40 μm and most preferably from 25 to 30 μm in thickness. The moisture vapor transmission rate of the polymer film of the wound dressing is preferably at least 300 g/m²/24 h, more particularly at least 1000 g/m²/24 h and most preferably at least 2000 g/m²/24 h (measured to DIN EN 13726, upright). In particularly preferred embodiments, these films have a moistureproof tacky edge portion. This edge portion ensures that the wound dressing can be applied to and fixed at its intended location. It is further ensured that liquid cannot escape between the film and the skin surrounding the area to be treated. Particularly preferable adhesives achieve in a thin add-on of 20 to 35 g/m² a moisture vapor transmission rate combined with the film of at least 400 g/m²/24 h and preferably of at least 1000 g/m²/24 h (measured to DIN EN 13726, upright).

The second layer of the wound dressing according to the present invention may be a backing layer with a lattice imprint, in which case the backing layer is in direct contact with the first layer. A lattice imprint may make it easier for the physician or medical personnel to assess a wound, for example to determine the wound size.

Here and hereinbelow, every content of an ingredient is to be understood in connection with the present invention—unless otherwise stated—as being in weight percent (wt %) based on the weight of the component comprising the ingredient.

The hydrogel matrix shall comprise more particularly from 54 to 60 wt % of the polyhydric alcohol propylene glycol. More particularly, the hydrogel matrix comprises from 56 to 58 wt % of propylene glycol and most preferably 57 wt % of propylene glycol. This alcohol is outstandingly useful as moisture donor and thus constitutes a care component for the skin surrounding the wound.

The hydrogel matrix shall further comprise in an amount altogether from 38 to 42 wt %, preferably from 39 to 41 wt % and more particularly 40 wt %, of a prepolymer having isophorone diisocyanate end groups and a diamine based on polyethylene oxide. It is also an essential feature of the invention that the ratio for reactive groups of isocyanate to amine groups of diamine be in the range from 1.25 to 1.35, since it is only then that the desired high absorption capacity and the advantageous pH on the part of the hydrogel matrix is obtained without adversely affecting other properties, for example the stability of the hydrogel. The ratio for reactive groups of isocyanate to amine groups of diamine shall preferably be in the range from 1.27 to 1.33 and more particularly in the range from 1.29 to 1.31. A ratio of 1.30 for reactive groups of isocyanate to amine groups of diamine is particularly advantageous.

It is further envisioned that the hydrogel matrix comprises at least one inorganic chloride. Sodium chloride, potassium chloride, magnesium chloride, calcium chloride or mixtures thereof are particularly suitable in this connection. These salts are particularly good at simulating the electrolyte mixture in wound serum. As a result, a hydrogel matrix comprising these salts provides similar osmotic conditions between hydrogel and wound exudate and thereby a climate that is particularly promotive of wound healing.

One possibility here is for the hydrogel matrix to comprise from 0 to 5 wt % of at least one inorganic chloride. More particularly, the hydrogel matrix comprises from 0.1 to 3 wt % of an inorganic chloride and most preferably from 0.5 to 1.5 wt % of an inorganic chloride. A hydrogel matrix containing 0.9 wt % of sodium chloride will prove very advantageous.

A hydrogel matrix comprising from 37 to 43 wt % of propylene glycol, altogether from 12 to 16.5 wt % of a prepolymer having isophorone diisocyanate end groups and of a diamine based on polyethylene oxide and from 0.5 to 1.5 wt % of an inorganic chloride, balance water, wherein the ratio for reactive groups of isocyanate to amine groups of diamine shall be in the range from 1.25 to 1.35, has a free absorbency A₃ (measured to DIN EN 13726-1 (2002)) of at least 1 g/g and at most 10 g/g. The hydrogel matrix provides a nonirritant, liquid-absorbing, cushioning, skinlike medium that affords protection from bacteria, and thus is particularly useful as a wound contact layer.

According to the present invention, the first layer is preferably a wound contact layer. A wound contact layer herein is a layer which can be in direct contact with the wound during use. The hydrogel matrix here may perform different functions in relation to the wound to be treated. The hydrogel matrix acting as wound contact layer can provide a milieu which supports wound healing. To this end, the hydrogel matrix can provide the wound with moisture and at the same time absorb excess liquid. A milieu supporting wound healing is also provided by a pH close to neutral, for example by a pH of 7.5. The hydrogel matrix can further protect the sensitive wound surface from shearing forces. Finally, the hydrogel matrix can endow the wound dressing with a certain degree of tack to the wound site. This facilitates applying the bandage.

The first layer, comprising a hydrogel matrix, may in addition to the hydrogel matrix further comprise a further layer, for example a polymer film, a hydrocolloid matrix, a polymer mesh, a nonwoven or an adhesive. The at least one further layer can be on the side which in use is remote from the wound. In this embodiment of the invention, the wound contact layer is formed by the at least one further layer. In a further embodiment of the invention, the further layer can be situated between the first layer and the second layer.

In an advantageous embodiment of the invention, the wound dressing comprises a first layer as wound contact layer which comprises a hydrogel matrix, wherein the hydrogel matrix has a layer thickness of 0.4 to 5 mm, preferably 0.6 to 2.5 mm, more particularly 0.8 mm. Furthermore, layer thicknesses of 1.35 mm and of 2.0 mm have proved highly practicable, since a wound dressing comprising such a wound contact layer achieves a high absorption capacity for liquids. Hydrogel matrices having the aforementioned layer thicknesses, especially with layer thicknesses between 0.6 and 2.0 mm, are capable of imbibing a sufficient quantity of liquid/wound exudate without becoming too thick. These layer thicknesses can be the same at every point of the wound contact layer, or differ in various regions of the wound contact layer.

It is further preferable for the hydrogel matrix to comprise structures, especially linear or circular depressions, elevations, dimples or net patterns. These structures are preferably situated on that side surface of the hydrogel matrix which in use faces the wound. The structures preferably do not go all the way through the hydrogel matrix. The depth to which the structures extend preferably amounts to at least 2% and at most 50% and more particularly at least 5% and at most 25% of the thickness of the hydrogel matrix.

That side of the wound contact layer which in use faces the wound typically has a suitable covering/release foil applied to it in order that the wound contact layer may be protected. A siliconized polyethylene foil or a siliconized polyester foil can be used for this for example. The release foil may comprise suitable auxiliary means for facilitating detaching the release foil, for example grip strips formed by folding back.

In accordance with another further-developed concept of the present invention, the present invention also provides a wound dressing which includes a barrier layer between the hydrogel matrix and the second layer. Such a barrier layer may comprise a polymer film for example.

In a further-developed concept of the present invention, the present invention likewise also provides a multilayered wound dressing comprising a first absorbent layer as wound contact layer, which comprises a hydrogel matrix, a second layer as backing layer, which comprises a polymer film and a distributor layer. The distributor layer is more particularly connected to the wound contact layer. Such a wound dressing very advantageously includes, between the absorbent layer and the backing layer, a distributor layer which consists of a hydrophilic polyurethane foam. The distributor layer provides for distribution of the imbibed wound fluids over the entire area of the wound dressing especially above the absorbent layer, i.e., the wound fluids are imbibed not just in the z-direction (away from the wound, in the direction of the backing layer), but also in the x-y-direction (over the area of the wound dressing).

In a further advantageous embodiment, the multilayered wound dressing comprises a first layer, which comprises a hydrogel matrix as wound contact layer, and a second layer as backing layer, which first and second layers are in direct contact with each other. The backing layer in this embodiment may likewise utilize all the abovementioned materials.

In a further advantageous embodiment of the invention, the multilayered wound dressing further comprises, on the side which in use is remote from the wound, a wound documentation foil as described for example in German patent application DE102005027656.

It must be emphasized here that the herein recited features of the preferred or alternative incarnations of the inventions shall not be restricted to the individual preferences or alternatives. On the contrary, the combination of the incarnations or the combination of the individual features of the alternative forms must similarly count as belonging to an incarnation according to the present invention. Similarly, the invention must not be understood as being reduced by the following description of the drawings.

### DRAWINGS

FIG. 1 shows an embodiment of the inventive wound dressing (10) in cross section. The wound dressing (10) consists of a first absorbent layer (1) and a backing layer (2). The backing layer (2) can be in direct contact with the first layer (1) or be connected via an adhesive layer (not shown here). That side of the second layer (2) which in use is remote from the wound may have further applied to it (not shown here) an application foil which preferably comprises a peel-off aid. The first layer (1) is a wound contact layer composed of a hydrogel matrix. Customarily, the wound contact layer (1), which comprises a hydrogel matrix, will have applied to it (not shown here) a protective layer (release foil), for example a siliconized polyurethane foil. The second layer (2) in the embodiment shown in FIG. 1 is a backing layer composed of polyethylene or polyurethane for example. The multilayered wound dressing (10) may include an adhesive border, i.e., be fabricated as a so-called island dressing (not depicted here). An adhesive border can be configured for example as a backing foil (2) to which an adhesive has been applied and which extends beyond the absorbent layer (1) in the entire extent of the wound dressing.

### OPERATIVE EXAMPLE

### A) Producing the Hydrogel

The hydrogel is produced using the following aqueous solutions and components (component A, B, C):

Component A

| | |
|---|---|
| Propylene glycol USP30 | from Hedinger Aug. GmbH; Stuttgart, |
| (99.8%) | Germany |
| Aqua purificata | Water treatment plant |
| NaCl, purist, USP | from Hedinger Aug. GmbH; Stuttgart, |
| | Germany |

Component A is produced by combining the ingredients and stirring until the salt has completely dissolved. Component A is cooled down to 2° C.

Component B

| | |
|---|---|
| Jeffamin ED-2003 | from Huntsman; Everberg, Belgium |
| Aqua purificata | Water treatment plant |

The aqueous component B is produced by melting the solid Jeffamin at 56° C. and adding the melt to the initially charged water with stirring. Component B is cooled down to room temperature.

Component C

| | |
|---|---|
| Aquapol PI-13000-3 | from Carpenter; Richmond, USA |

Component C is brought to room temperature.

The prepared components A, B, and C are combined with each other in a homogeneous mixture by homogenization using a rotating mixing system and poured into the provided molds ideally without bubbles.

| Ex- | | | | | Pro- | | |
|---|---|---|---|---|---|---|---|
| peri- | Isocy- | Di- | | NCO/ | pylene | Wa- | |
| ment | anate ¹⁾ | amine ²⁾ | Polyurea ³⁾ | NH2 ⁴⁾ | glycol | ter | NaCl |
| 1.3 | 15.3 | 24.7 | 40 | 1.30 | 40 | 19.1 | 0.9 |
| 2.9 | 22.2 | 17.8 | 40 | 1.00 | 57 | 2.1 | 0.9 |
| 2.27 | 15.3 | 24.7 | 40 | 1.30 | 57 | 2.1 | 0.9 |
| 2.1.7 | 5.7 | 9.3 | 15 | 1.30 | 60 | 24.1 | 0.9 |

All particulars in wt % based on the composition.
¹⁾ isocyanate copolymer (Aquapol PI-13000-3), proportion
of reactive groups (NCO) 3.223%
²⁾ diamine (Jeffamine ED-2003, molecular weight 2000), amine content 0.9554 mol/g
³⁾ sum total of isocyanate and diamine
⁴⁾ ratio for reactive groups of isocyanate to amine groups of diamine

### B) Further Materials Used

The backing layer used is a water-impermeable polyurethane film (from Exopack-Wrexham, United Kingdom) 60 μm in thickness. This film is coated with an acrylate-based pressure sensitive adhesive in a layer 30 μm in thickness. The film has a moisture vapor transmission rate MVTR (upright) of 1100 g/m²/24 h (DIN EN 13726-1).

The release foil used is a one-sidedly siliconized LDPE foil of the type SKV1002 (from Deku Kunstsatofffabrik, Pommelsbrunn, Germany) 70 μm in thickness. The siliconized side is oriented toward the gel.

### C) Producing the Wound Dressings

The wound dressings (specimens) are fabricated by hand in accordance with the following sequence:
1. To produce an inventive wound dressing, a hydrogel composition as described above under A) is provided as first layer. The hydrogel has to be further processed immediately after the comixing and homogenizing. To form the hydrogel matrix, the gel is poured onto the polyurethane foil.
2. The gel is distributed with a squeegee such that the gel layer has a height of 0.8 mm.
3. After polymerization, i.e., after about 10 minutes, the hydrogel is laid onto the prepared release foil (the siliconized side is oriented toward the gel), so that the side directed toward the wound is protected.
4. Wound dressings having an edge length of 10×10 cm are die cut out of the multi-ply assembly of materials.

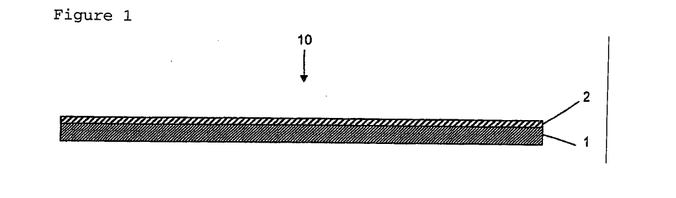

The wound dressing thus produced has the construction described using FIG. 1, although FIG. 1 does not show a release liner.

### D) Methods of Measurement

Absorptive capacity in respect of demineralized water is determined in line with EN13726-1. The measurement takes place at 37° C. Sample pieces'2.5×2.5 cm in size are cut out of the middle of the hydrogel layer. Any covering film is peeled off and removed. The samples are weighed into a glass beaker. Demineralized water is then added in 40 times the amount. The glass beaker is covered with a watch glass. After 48 h the samples are reweighed. Water imbibition is computed in g of water per g of gel piece (g/g).

The pH of a hydrogel matrix is determined in connection with the present invention by laying the hydrogel into water and measuring the pH of the solution. The measurement is carried out at room temperature (20° C.). Sample pieces 2.5×2.5 cm in size are cut out of the middle of the hydrogel layer. Any covering film is peeled off and removed. The samples are weighed into a glass beaker. Demineralized water is then added in an amount of 12.5 ml. The glass beaker is covered with a watch glass. After a period of 24 h the sample is removed from the solution. A pH electrode is then dipped into the solution temperature-controlled to 20° C. The pH is read off as soon as the displayed numerical value remains stable.

The tack of the wound dressing to intact skin was estimated. Commercially available products were put into 5 categories by one person. A tack of 1 on the scale indicates that the wound dressing does not adhere to healthy skin, while a tack of 5 indicates a very high level of tack. It was assessed on the basis of the subjective impression of one person by comparison with the tack of the prepared wound dressings as perceived between the index finger and the gel.

### E) Properties of Wound Dressings

| | pH³⁾ | Absorbency⁴⁾ | Tack |
|---|---|---|---|
| wound dressing¹⁾ with hydrogel from | 7.51 | 3.33 g/g | 1 |
| experiment 1.3 | | | |
| wound dressing¹⁾ with hydrogel from | 8.99 | 9.56 g/g | 4 |
| experiment 2.9 | | | |
| wound dressing¹⁾ with hydrogel from | 7.30 | 5.19 g/g | 2 |
| experiment 2.27 | | | |
| hydrogel from experiment 2.1.7 | (gel does not cure; | | |
| | measurement impossible) | | |
| Hydrosorb wound dressing²⁾ | 8.06 | 2.00 | 1 |

The Hydrosorb wound dressing (Paul Hartmann AG) contains a hydrogel having a layer thickness of 2.0 mm. The hydrogel used for producing Hydrosorb comprises 11.2 wt % of Jeffamin ED-2003, 6.8 wt % of Aquapol PI-13000-3, 18 wt % of propylene glycol and 63 wt % of water, 1 wt % of NaCl. The ratio for reactive groups of isocyanate to amine groups of diamine therein is 1.23.
¹⁾hydrogel thickness 0.8 mm
²⁾hydrogel thickness 2.0 mm
³⁾pH determination as described under D)
⁴⁾absorbency of demineralized water within 48 h was determined in line with EN13726-1 as described under D).

The wound dressing with the hydrogel from experiment 1.3 has a pH of 7.51, which is promotive of wound healing. Absorption capacity is superior to that of the prior art hydrogel. The wound dressing has no surface tack to skin.

The wound dressing with the hydrogel from experiment 2.9 has a very alkaline pH of about 9, which is unacceptable for wound treatment. The wound dressing further exhibits very high absorbency and a tack of 4, i.e., the wound dressing adheres firmly to the skin and is easy to remove. Tack is in the desired range. Despite high absorbency and advantageous tack, the wound dressing is not suitable for treating tissue defects in a manner promotive of wound healing owing to the unfavorable pH.

The wound dressing with the hydrogel from experiment 2.27 has a desirable pH of 7.30 for wound treatment, i.e., a near-neutral pH. The wound dressing further exhibits high absorbency for fluids and a tack of 2, i.e., a low tack for skin. The wound dressing overall exhibits a very advantageous combination of pH and absorbency.

The composition tested in experiment 2.1.7 could not be used to obtain a stable hydrogel, since the gel did not fully cure. It was accordingly not possible to produce a multilayered wound dressing therefrom.

1. A multilayered wound dressing comprising
a) a first layer as absorbent layer comprising a hydrogel matrix, and
b) at least one second layer, applied atop the first layer on the wound-remote side,
characterized in that the hydrogel matrix comprises from 54 to 60 wt % of propylene glycol, altogether from 38 to 42 wt % of a prepolymer having isophorone diisocyanate end groups and of a diamine based on polyethylene oxide and from 0 to 5 wt % of an inorganic chloride, balance water, wherein the ratio for reactive groups of isocyanate to amine groups of diamine shall be in the range from 1.25 to 1.35.

2. The wound dressing according to claim 1, wherein the hydrogel matrix comprises from 56 to 58 wt % of propylene glycol and altogether from 39 to 41 wt % of a prepolymer having isophorone diisocyanate end groups and of a diamine based on polyethylene oxide.

3. The wound dressing according to claim 1, wherein the hydrogel matrix comprises 57 wt % of propylene glycol, 24.7 wt % of a prepolymer having isophorone diisocyanate end groups, 15.3 wt % of a diamine based on polyethylene oxide, 0.9 wt % of sodium chloride, and balance water.

4. The wound dressing according to claim 1, wherein the second layer is a backing layer.

5. The wound dressing according to claim 1, wherein the second layer comprises films or foams of polyurethane, polyetherurethane, polyesterurethane, polyether-polyamide copolymers, polyacrylate, polymethacrylate or mixtures thereof.

6. The wound dressing according to claim 1, wherein the second layer comprises an adhesive-coated polymer foil with a lattice imprint and in direct contact with the first layer.

7. The wound dressing according to claim 6, wherein the wound dressing comprises a further polyurethane foil on that side of the wound dressing which in use is remote from the wound.

8. The wound dressing according to claim 1, wherein the wound dressing further comprises a release foil on that side of the wound dressing which in use faces the wound.

9. The wound dressing according to claim 1, wherein the wound dressing further comprises an application foil on that side of the wound dressing which in use is remote from the wound.

10. The wound dressing according to claim 1, wherein the wound dressing further comprises a documentation foil.
